(19) United States
(12) Patent Application Publication
(10) Pub. No.: US 2007/0074971 A1
(43) Pub. Date: Apr. 5, 2007

(54) Sensor for ascorbic acid or a salt thereof

(76) Inventors: Anne Louise Atkinson, Wavendon (GB); Karl John Hunter, Sharnbrook (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 10/549,792

(22) PCT Filed: Mar. 4, 2004
(86) PCT No.: PCT/EP04/02191

§ 371 Date: Oct. 24, 2006

(30) Foreign Application Priority Data
Mar. 21, 2003 (EP) 0306567.9

(51) Int. Cl. G01N 33/487

(52) U.S. Cl. 204/403.01

(57) ABSTRACT

A sensor for detecting ascorbic acid or a salt thereof is provided, the sensor having sensor means for detecting ascorbic acid or a salt thereof in a liquid sample and buffer means for buffering the sample before and/or at the time that the sample contacts the sensor means, said buffer means having two zones, a first zone for buffering the sample to a pH in the range of from 5.5 to 8, preferably from 6.5 to 7.5 and a second zone for receiving sample which has passed through the first zone and for buffering to a pH in the range from 1 to 5.

### FIELD OF THE INVENTION

The present invention relates to the field of sensors for the detection of ascorbic acid or a salt thereof.

### BACKGROUND OF THE INVENTION

Vitamin C in the form of ascorbic acid or a salt thereof is known to fulfil a wide range of roles in maintaining health and reducing some negative effects of ageing. It would therefore be desirable if an individual could monitor his or her vitamin C intake to determine whether it is at an optimum level.

For home evaluation of vitamin C levels, it is especially convenient to test urine samples. However, with a urine sample, for several forms of assay, uric acid is a strong interferent.

### SUMMARY OF THE INVENTION

We have now found that a sensor in which the sample is buffered sequentially to different pH values can reduce this problem. Moreover, it is also appropriate or necessary for certain kinds of assay to function with adequate sensitivity. Thus, the present invention may also be adapted to use with other biosamples such as blood or saliva or with other liquid samples such as fruit juice.

Accordingly, the present invention provides a sensor for ascorbic acid or a salt thereof, the sensor comprising sensor means for detecting ascorbic acid or a salt thereof in a liquid sample and buffer means for buffering the sample before and/or at the time that the sample contacts the sensor means, said buffer means comprising two zones, a first zone comprising filter means and means for buffering the sample to a pH in the range of from 5.5 to 8, preferably from 6.5 to 7.5 and a second zone for receiving sample which has passed through the first zone and for buffering to a pH in the range from 1 to 5.

The present invention also provides a method of detecting ascorbic acid or a salt thereof, which method comprises contacting a sensor of the invention with a liquid sample. Typically said sample contains an interferent such as uric acid.

### DETAILED DESCRIPTION OF THE INVENTION

The two pH buffering zones in the sensor according to the present invention are provided for two different reasons.

The first zone buffers to a pH in the range of from 5.5 to 8, preferably from 6.5 to 7, to enable the filter means to reduce the level of uric acid interferent in urine without significantly reducing the level of the ascorbic acid or ascorbate. It can also reduce the level of some other interferents in urine and other interferents in other liquid samples. It may for example be supported on, or impregnated into, the said filter means.

The second zone buffers the sample after passage through the first zone to render the sample at optimum pH for sensing the ascorbic acid or salt thereof. It may for example be supported on, or impregnated into, a filter means and/or be contiguous with the sensor means, especially when the latter comprises electrodes of an electrochemical sensor.

Suitable buffers are well known in the art. For example, for the first zone, a buffer substance may be selected from sodium phosphate, HEPES or TRIS (or mixtures thereof). For the second zone, for example, a buffer substance may be selected from sodium formate, sodium acetate, oxalic acid, or phthalic acid (or mixtures thereof).

The sensor means may, for example, comprise a calorimetric sensor or an electrochemical sensor. In the case of the electrochemical sensor, the second zone should buffer to a pH in the range of from 1 to 4.5, preferably from 2.5 to 4. In the case of a calorimetric sensor, the second zone should buffer to a pH in the range of from 3.5 to 5, preferably from 3.7 to 4.8. Preferably, the buffer means comprises respective buffer substances impregnated into filter means or supported on a support, which may itself be filter means.

One such suitable arrangement is to provide two separate filter members positioned so that in use, one is situated above the other. In this way, the liquid sample can pass through the first filter member to fall onto or be absorbed onto the second filter member. In an alternative arrangement, the two zones may comprise separate zones of a substantially elongate absorbent strip. In that case, the sample may be absorbed onto one end with the sensor means located at the other end, the two zones being located therebetween. In either of these arrangements, or in any other arrangement, the two zones may abut or touch one another or may be mutually separated.

Although the pH buffering of the first zone reduces uric acid or in some cases, one or more other interferents, the filter means may also help to reduce interferent level in one or more other ways.

For example, the arrangement of separate filter members through which the sample falls, can trap solid material. On the other hand, if the filter means comprises a strip, this may provide spatial/temporal separation between ascorbic acid/ascorbate and interferent(s) in the manner of a thin layer chromatograph.

In some preferred embodiments, the first and/or second zone may be further impregnated with one or more additional agents for reducing the level of one or more interferents in the sample. The additional agent or agents for reducing one or more interferents may also be provided elsewhere in the overall sensor device. For example, in those embodiments which comprise a substantially elongate absorbent strip, they may be supported on or impregnated into another part or spread across a wide area of the strip (optionally also encompassing one or both pH buffering zones). In the case of filter members arranged one above another, such an additional material may be impregnated into or supported on one or more other filter members disposed above or below either filter member acting as a buffering zone.

In the case of a strip kind of construction, the absorbent material may for example comprise filter paper or nitrocellulose. Other suitable filter materials for use in the kind of sensor device where a plurality of filter members are arranged above one another, as well as filter paper or nitrocellulose, there may be used one or more of aluminium oxide (Al₂O₃), amino-silica, cellulose, cyano-silica, hydroxyapatite (Ca₅(PO₄)₃OH), nitrocellulose, phenyl-silica, polyamide and silica (SiO₂).

Additionally or alternatively the second zone may be further impregnated with or support a reagent for reacting with the ascorbic acid or a salt thereof. One or more reaction products of this reaction may be detected by suitable means, e.g. visually or with a colorimetric sensor or an electrochemical sensor. Such a reagent may additionally or alternatively be located in any of the other locations specified above with regard to the optional additional agents for reducing the level of one or more interferents. A non-exhaustive list of such reagents comprises one or more of ferrozine, 2,4-dinitrophenylhydrazone, 2,6-dichlorophenolindolphenol, nitroblue tetrazolium, 2,4,6-tripyridyl-S-triazine and 2,2′-dipyridine.

A suitable electrochemical sensor is a redox sensor. Redox species have inherent electrochemical activity and are therefore capable of exchanging electrons directly with a working electrode to produce an electrochemical signal.

For the purpose of the present invention particular attention is directed to a device which overcomes the associated problem of interferents with strong electrochemistry such as uric acid, proteins and paracetamol.

The electrode structure preferably comprises a working electrode, a counter electrode and a reference electrode, however it is recognised that the reference and counter electrodes may be combined in some circumstances e.g. when the measurement of current is small (˜nA).

The counter electrode should be of sufficient size in relation to the working electrode so that the electrochemical reaction at the charge transfer interface is not limited. Preferably the counter electrode is at least 5 times the size of the working electrode, more preferably at least 10 times the size.

The present invention will now be explained in more detail by way of the following description of preferred embodiments and with reference to the accompanying drawings:-

### BRIEF DESCRIPTION OF THE DRAWINGS

FIG. 1 shows a first embodiment of a sensor according to the present invention; and

FIG. 2 shows a second embodiment of a sensor according to the present invention.

### DESCRIPTION OF PREFERRED EMBODIMENTS

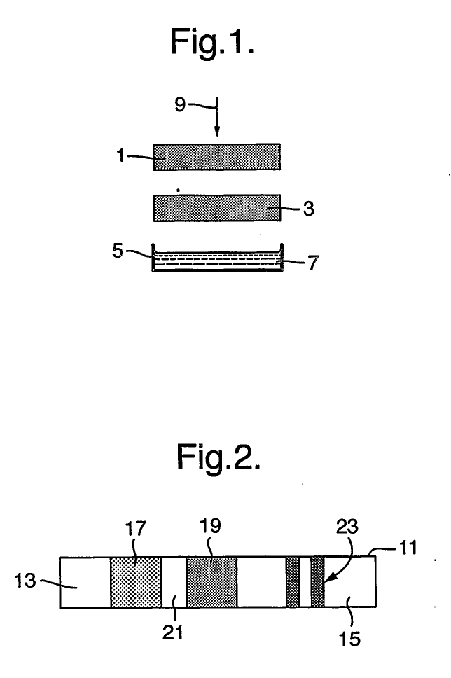

FIG. 1 shows an arrangement of a sensor according to the present invention. As shown in this figure, a first filter member 1 is disposed above a second filter member 3 which is in turn situated above a cuvette 5 in which a treated liquid sample 7 is collected.

The first filter member 1 is made of cellulose powder and is impregnated with HEPES to buffer sample passing therethrough, to a pH of approximately 6.8.

The second filter member 3 is also made of cellulose powder and is impregnated with ferrozine which is capable of reacting with ascorbic acid or a salt thereof to undergo a colour change. The second filter member 3 is also impregnated with sodium formate to buffer sample to a pH of approximately 4.0.

As denoted by arrow 9, a urinary sample is dripped onto the upper filter member 1 to be buffered to a pH of approximately 6.8 and for at least partial removal of uric acid and protein interferents.

The sample then falls onto the lower filter member 3 to be buffered to a pH of approximately 4.0 and to interact with the reagent whereupon it falls into the cuvette 5 to form a reagent/ascorbic acid or ascorbate solution 7 the colour of which (due to the ferrozine), denotes whether or not adequate vitamin C content is detected in the urine.

Turning to FIG. 2, there is shown an absorbent test strip 11 which comprises a first or sampling end 13 and a second or sensing end 15. In this embodiment, the urine sample is dropped onto the first sampling end 13.

A first zone 17 adjacent the sampling end 13 is impregnated with the same materials as the first filter member 1 in the previous embodiment.

On the other side of the first zone 17 is located a second zone 19 impregnated with the same materials as the second filter member 3 of the first embodiment. The first and second zones 17, 19 are mutually separated by a non-impregnated region 21.

On the other side of the second zone 19 from the first zone 17, adjacent the sensing end 15, is located a printed electrochemical electrode structure 23 for determining ascorbic acid or ascorbate content. The output of the electrodes is evaluated using conventional circuitry (not shown).

The functions of the two zones 17, 19 are analogous to that of the first and second filter members 1, 3 of the first embodiment.

1. A sensor for ascorbic acid or a salt thereof, the sensor comprising sensor means for detecting ascorbic acid or a salt thereof in a liquid sample and buffer means for buffering the sample before and/or at the time that the sample contacts the sensor means, said buffer means comprising two zones, a first zone comprising filter means and means for buffering the sample to a pH in the range of from 5.5 to 8 and a second zone for receiving sample which has passed through the first zone and for buffering to a pH in the range from 1 to 5.

2. A sensor according to claim 1, wherein the buffer means comprises buffer substances impregnated into or supported on filter means.

3. A sensor according to claim 2, wherein the two zones comprise two separate filter members of the filter means, arranged so to be one above the other when the sensor is in use.

4. A sensor according to claim 2, wherein the two zones comprise zones of a substantially elongate absorbent filter strip.

5. A sensor according to claim 1, further comprising one or more agents for reducing the level of one or more interferents.

6. A sensor according to claim 5, wherein at least one of said one or more agents is located in at least one of the two zones.

7. A sensor according to claim 1, wherein the second zone further comprises a reagent for reacting with ascorbic acid or a salt thereof or the reagent is located downstream of the second zone.

8. A sensor according to claim 1, wherein the sensor means comprises a calorimetric sensor.

9. A sensor according to claim 8, wherein the second zone is adapted to buffer the sample to a pH in the range of from 3.5 to 5.

10. A sensor according to claim 1, wherein the sensor means comprises an electrochemical sensor means.

11. A sensor according to claim 10, wherein the second zone is adapted to buffer the sample to a pH in the range of from 1 to 4.5.

12. A sensor according to claim 10, wherein the electrode arrangement is printed on the absorbent filter strip.

13. A sensor according to claim 10, wherein the electrochemical sensor means is at least partly located in the second zone.

14. A method of detecting ascorbic acid or a salt thereof, which method comprises contacting a sensor according to claim 1 with a liquid sample such that the sample passes through said first zone prior to said second zone.
